(19) United States
(12) Patent Application Publication
(10) Pub. No.: US 2022/0068446 A1
(43) Pub. Date: Mar. 3, 2022

(54) UTILIZATION OF MEDICAL DATA ACROSS ORGANIZATIONAL BOUNDARIES

(71) Applicant: Siemens Healthcare GmbH, Erlangen (DE)

(72) Inventor: Asmir VODENCAREVIC, Fuerth (DE)

(73) Assignee: Siemens Healthcare GmbH, Erlangen (DE)

(21) Appl. No.: 17/412,455

(22) Filed: Aug. 26, 2021

(30) Foreign Application Priority Data
Sep. 1, 2020 (DE) 10 2020 210 998.2

(51) Int. Cl. G16H 10/60; G06K 9/62; G06N 20/00; G06F 16/25

(52) U.S. Cl. CPC G16H 10/60; G06K 9/6223; G06F 16/252; G06K 9/6256; G06N 20/00; G06K 9/6262

(57) ABSTRACT

Methods and apparatuses are for a medical dataset stored locally within a first facility and including a number of original individual datasets assigned to real existing patients and including original values for one or more higher-ranking variables. An embodiment of the method includes creation of a synthetic dataset based on the medical dataset, the synthetic dataset including a number of synthetic individual datasets including synthetic values for the same higher-ranking variables as the medical dataset, not relatable to an original existing patient, the creation being undertaken locally within the first facility by application of a sampling function to the medical data; and transfer of the synthetic dataset from the first facility to a central unit outside the first facility. The synthetic dataset is utilizable within the central unit.

### PRIORITY STATEMENT

The present application hereby claims priority under 35 U.S.C. § 119 to German patent application number DE 102020210998.2 filed Sep. 1, 2020, the entire contents of which are hereby incorporated herein by reference.

### FIELD

Example embodiments of the invention generally relate to methods and systems for utilization of medical data in a “distributed environment” across organizational boundaries, in particular taking into account data security aspects.

### BACKGROUND

The key to the development of artificial intelligence and machine learning lies in the availability of data, which is indispensable for the training and validation of intelligent algorithms.

In many areas however the exchange of data is subject to restrictive limitations. Thus personal data in particular can be subject to data protection regulations, which set strict limits on the utilization of information. The passing on of such data frequently requires its anonymization or at least its pseudonymization. Since the data structures can be very different from provider to provider, there are hardly any uniform solutions for this however. Moreover, depending on the jurisdiction, the general legal conditions can differ considerably. The systematic anonymization of data is therefore a time- and cost-intensive task. Moreover there is the risk of a possible re-identification of personal data, which can entail significant legal and financial consequences. Many facilities or organizations that have personal data available to them therefore adopt a critical attitude to passing on this data for research and development purposes.

This applies all the more to the medical field. On the one hand access to patient data is a prerequisite for the development of advanced algorithms based on artificial intelligence. On the other hand such patient data is particularly sensitive. In this field in particular any incoherence regularly attracts great attention and is associated with a corresponding loss of reputation for the facility involved. Many facilities therefore insist that patient data remains within the facility concerned and does not leave the facility. Since many artificial intelligence systems are developed and validated externally and rely on as many datasets as possible from different facilities, this represents a major restriction for research and development.

What is more, the data as such by now represents a value. Permanent access to a valuable dataset, by purchasing it for example, can therefore be associated with high costs. For these reasons access to a development partner's data is frequently limited to the duration of the collaboration. Moreover personal data is regularly deleted and then is no longer available.

### SUMMARY

The inventors have discovered that all this makes a reproducible and sustained development of artificial intelligence systems more difficult. New ideas cannot be tested and subsequent validations or quality audits are no longer possible. The inventors have discovered that, moreover, a disclosure or exchange with other developers is frequently restricted.

At least one embodiment of the present invention therefore provides methods and/or systems with which a lasting utilization of medical data can be ensured. In this case it should be made possible in particular to exchange information residing within the medical data between facilities across organizational boundaries while taking into account relevant data protection policies.

Embodiments of the invention are directed to, a method, an apparatus, a computer program product or a computer-readable storage medium. Advantageous developments are specified in the claims.

In accordance with one form of embodiment of the invention a computer-implemented method for creating a synthetic dataset based on a medical dataset is provided. The method has a number of steps. A first step is directed to the provision of a medical dataset. The medical dataset has a number of original individual datasets, which are assigned to real existing patients and have original values for one or more higher-ranking variables. A further step is directed to the creation of a synthetic dataset based on the medical dataset, wherein the synthetic dataset has a number of synthetic individual datasets, which have synthetic values for at least some of the higher-ranking variables of the medical dataset, but cannot be related back to a real existing patient. In a creation step the synthetic dataset is created by application of a sampling function to the medical dataset. In this case the sampling function is embodied to create the synthetic dataset by sampling the entire medical dataset while replacing all original values.

In accordance with a further form of embodiment a computer-implemented method for utilization of a medical dataset is provided. The medical dataset in this case is stored locally within a first facility. The medical dataset has a number of original individual datasets, which are assigned to real existing patients and have original values for one or more higher-ranking variables. The method comprises a number of steps. One step is directed to the (local) creation of a synthetic dataset through application of the sampling function to the medical dataset, wherein the step of creation is undertaken locally within the first facility, and the synthetic dataset features a number of synthetic individual datasets, which synthetic individual datasets have synthetic values for the same higher-ranking variables as the medical dataset, but cannot be related back to a real existing patient. A further step is directed to the transfer of the synthetic dataset from the first facility to a second facility outside the first facility different from the first facility. A further step is directed to a utilization of the synthetic dataset within the second facility.

One method for supervised learning can appear as follows in accordance with one form of embodiment. To this end a computer-implemented method for provision of a trained function for creating a synthetic dataset based on a medical dataset is provided. The method has the following steps:

provision of training input data, wherein the training input data represents a medical dataset;

provision of training output data, wherein the training output data represents a desired synthetic dataset;

creation of a synthetic dataset by application of the trained function to the training input data;

comparison of the created synthetic dataset with the training output data;

adaptation of the trained function based upon the comparison.

The invention further relates, in a further embodiment, to a computer program product, which comprises a program and is able to be loaded directly into a memory of a programmable computing unit and has program means, e.g. libraries and auxiliary functions, for carrying out a method of an embodiment for creating a synthetic dataset for cross-facility utilization of medical datasets, when the computer program product is executed.

The invention further relates, in a further embodiment, to a computer-implemented method for a medical dataset, comprising:

storing the medical dataset within a first facility, the medical dataset including a number of original individual datasets assigned to real existing patients and including original values for one or more higher-ranking variables;

creating a synthetic dataset based on the medical dataset, each synthetic individual dataset of the number of synthetic individual datasets including synthetic values for same higher-ranking variables as the one or more higher-ranking variables medical dataset, not relating back to a real existing patient, wherein the creating is undertaken within the first facility by application of a sampling function to the medical data; and

transferring the synthetic dataset from the first facility to a central unit outside the first facility, the synthetic dataset being utilizable within the central unit.

The invention further relates, in a further embodiment, to a system for a medical dataset, a first facility storing the medical dataset, the medical dataset including a number of original individual datasets assigned to real existing patients and including original values for one or more higher-ranking variables, the system comprising:

a computing unit, located outside the first facility; and

an interface for communication between the computing unit and the first facility, wherein

the computing unit is embodied:
to induce a local creation of a synthetic dataset in the first facility via the interface, the synthetic dataset including a number of synthetic individual datasets, each synthetic individual dataset of the number of synthetic individual datasets including synthetic values for same higher-ranking variables as the one or more higher-ranking variables medical dataset, not relating back to a real existing patient;
to receive the synthetic dataset from the first facility via the interface; and
to utilize the synthetic dataset outside the first facility.

The invention further relates, in a further embodiment, to a non-transitory computer program product, including a program, directly loadable into a memory of a programmable computing unit of a processing unit, the program including program segments for carrying out the method of an embodiment when the program is executed in the computing unit of the processing unit.

The invention further relates, in a further embodiment, to a non-transitory computer-readable memory medium, storing readable and executable program sections for carrying out the method of an embodiment when the program sections are executed by at least one of a determination system and training system.

### BRIEF DESCRIPTION OF THE DRAWINGS

Example embodiments of the invention are explained in greater detail below with the aid of drawings. In the drawings, in schematic and simplified diagrams:

FIG. 1 shows a system for utilization of medical datasets in accordance with one form of embodiment;

FIG. 2 shows a flow diagram of a method for utilization of medical datasets in accordance with one form of embodiment;

FIG. 3 an illustration of the interactions of the system components during a utilization of medical datasets in accordance with one form of embodiment;

FIG. 4 shows a schematic outline relating to the processing of data during creation of synthetic datasets in accordance with one form of embodiment;

FIG. 5 shows a flow diagram of a method for creating synthetic datasets based upon medical datasets in accordance with one form of embodiment;

FIG. 6 shows a flow diagram of a method for optimizing the creation of synthetic datasets based upon medical datasets in accordance with one form of embodiment; and

FIG. 7 shows a flow diagram of a method for handling longitudinal data during the creation of synthetic datasets based upon medical datasets in accordance with one form of embodiment.

Parts and variables corresponding to one another are always labeled with the same reference characters in all figures. Modifications cited in this context can be combined with one another in each case in order to produce new forms of embodiment.

### DETAILED DESCRIPTION OF THE EXAMPLE EMBODIMENTS

The drawings are to be regarded as being schematic representations and elements illustrated in the drawings are not necessarily shown to scale. Rather, the various elements are represented such that their function and general purpose become apparent to a person skilled in the art. Any connection or coupling between functional blocks, devices, components, or other physical or functional units shown in the drawings or described herein may also be implemented by an indirect connection or coupling. A coupling between components may also be established over a wireless connection. Functional blocks may be implemented in hardware, firmware, software, or a combination thereof.

Various example embodiments will now be described more fully with reference to the accompanying drawings in which only some example embodiments are shown. Specific structural and functional details disclosed herein are merely representative for purposes of describing example embodiments. Example embodiments, however, may be embodied in various different forms, and should not be construed as being limited to only the illustrated embodiments. Rather, the illustrated embodiments are provided as examples so that this disclosure will be thorough and complete, and will fully convey the concepts of this disclosure to those skilled in the art. Accordingly, known processes, elements, and techniques, may not be described with respect to some example embodiments. Unless otherwise noted, like reference characters denote like elements throughout the attached drawings and written description, and thus descriptions will not be repeated. At least one embodiment of the present invention, however, may be embodied in many alternate forms and should not be construed as limited to only the example embodiments set forth herein.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments of the present invention. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “connected,” “engaged,” “interfaced,” and “coupled.” Unless explicitly described as being “direct,” when a relationship between first and second elements is described in the above disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments of the invention. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

When an element is referred to as being “on,” “connected to,” “coupled to,” or “adjacent to,” another element, the element may be directly on, connected to, coupled to, or adjacent to, the other element, or one or more other intervening elements may be present. In contrast, when an element is referred to as being “directly on,” “directly connected to,” “directly coupled to,” or “immediately adjacent to,” another element there are no intervening elements present.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

Before discussing example embodiments in more detail, it is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments of the present invention. This invention may, however, be embodied in many alternate forms and should not be construed as limited to only the embodiments set forth herein.

Units and/or devices according to one or more example embodiments may be implemented using hardware, software, and/or a combination thereof. For example, hardware devices may be implemented using processing circuity such as, but not limited to, a processor, Central Processing Unit (CPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind, however, that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one embodiment of the invention relates to the non-transitory computer-readable storage medium including electronically readable control information (procesor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

Embodiments are described below both with regard to the apparatuses and also with regard to the methods. Features, advantages or alternate forms of embodiment mentioned here are likewise to be transferred to the other subject matter and vice versa. In other words the physical claims (which are directed to an apparatus for example) can also be developed with the features that are described or claimed in conjunction with a method. The corresponding functional features of the method are embodied in such cases by corresponding physical modules.

Embodiments are furthermore described both with regard to methods and apparatuses for creating a synthetic dataset or for utilization of a medical dataset and also with regard to methods and apparatuses for adapting or optimizing trained functions. Features and alternate forms of embodiment of data structures and/or functions for methods and apparatuses for determination are transferred to similar data structures and/or functions for methods and apparatuses for adapting/optimizing. Similar data structures here can in particular be identified by the use of the prefix “training”. Furthermore the trained functions used in methods and apparatuses for creating a synthetic dataset or for utilization of a medical dataset can have been adapted and/or provided in particular by methods for adapting trained functions.

The features of the forms of embodiment presented below, provided they are not mutually exclusive, can be combined with one another in order to form new forms of embodiment.

In accordance with one form of embodiment of the invention a computer-implemented method for creating a synthetic dataset based on a medical dataset is provided. The method has a number of steps. A first step is directed to the provision of a medical dataset. The medical dataset has a number of original individual datasets, which are assigned to real existing patients and have original values for one or more higher-ranking variables. A further step is directed to the creation of a synthetic dataset based on the medical dataset, wherein the synthetic dataset has a number of synthetic individual datasets, which have synthetic values for at least some of the higher-ranking variables of the medical dataset, but cannot be related back to a real existing patient. In a creation step the synthetic dataset is created by application of a sampling function to the medical dataset. In this case the sampling function is embodied to create the synthetic dataset by sampling the entire medical dataset while replacing all original values.

The medical dataset in particular has personal data for one or more patients. The medical dataset can be construed as a totality of the available data for a patient cohort. A patient cohort can be defined in this case by the fact that it belongs to one or more case groups. For example all patients, who have been or are being treated in an organization or facility for a specific illness can be grouped together into a patient cohort. Accordingly the medical dataset in each case has a number of individual datasets (also called “original individual datasets” or “real individual datasets” below), which in their turn are assigned to patients in each case. The original individual datasets can be related back for example to an examination of a patient. In this case a number of different original individual datasets can also be assigned to one patient, which can e.g. relate to examinations of the patient carried out at different times.

Each original individual dataset has original values for one or more higher-ranking variables. The original values are thus likewise assigned to real existing patients. The original values can feature measured values, such as e.g. laboratory values, vital values, or examination parameters (e.g. number of painful joints), personal information about patients, information about medication etc. of the patient, which has been obtained during an examination of the patient for example. The higher-ranking variables can further relate to values, variables and/or features that have been extracted from text or image data (which in their turn are based on an examination of the patient).

The original values can be obtained automatically and/or manually and be supplied or have been supplied to the medical dataset. Text data in such cases can be pathological and/or radiological findings. Image data can in particular be medical image data (e.g. radiology or histopathology images).

The extraction of such values, variables and/or features can have been undertaken manually by a doctor during an investigation or automatically by automated image processing and text recognition algorithms. Higher-ranking variables can be construed as a category or type of the original values. One or more higher-ranking variables of the medical dataset can be numeric variables, which relate to numeric original values. These can for example comprise an ID, an age, a time at which the individual dataset was obtained, one or more inflammation parameters or the blood pressure of a patient. As well as this one or more higher-ranking variables can be categorical variables, which are related to non-numeric values. Such non-numeric values for example can include simple binary expressions such as ‘yes’ or ‘no’, or classifications such as ‘low’, ‘medium’ or ‘high’. The original individual datasets in this case can address higher-ranking variables. The original individual datasets can in particular be part of an Electronic Medical Record (EMR) of the respective patient.

‘Provision’ with regard to the medical dataset can mean that the medical dataset or the original individual datasets are able to be retrieved or are retrieved from a corresponding database in which they are archived, and/or are loaded or are able to be loaded into a computing unit, in order to create the synthetic dataset in the computing unit. Such databases can for example be part of one or more medical information systems, such as for example a Hospital Information System (HIS), a Radiology Information System (RIS), Laboratory Information Systems (LIS), a Cardiovascular Information System (CVIS) and/or a Picture Archiving and Communicating System (PACS).

The synthetic dataset is created based on the medical dataset. The synthetic dataset can be understood as a type of ‘map’ of the medical dataset, in which the assignment or the ability to be assigned to real existing patients has been eliminated. The synthetic individual datasets can consequently no longer be assigned to real existing patients of the medical dataset. In such cases the synthetic dataset merely involves an anonymized or pseudonymized version of the medical dataset. Instead the synthetic dataset features synthetic individual datasets with synthetic values, which have been created based on original individual datasets. Merely anonymized datasets on the other hand would still contain the original individual datasets (then in an anonymized form). While with merely anonymized datasets an identification of the patient is thus still possible, through a data reconciliation for example, this can be excluded by the creation of synthetic data.

The synthetic dataset in this case is created with a sampling function, which is applied to the medical dataset. The sampling function can be construed in particular as a computer program product, which is embodied for creating a synthetic dataset based on real medical data. The sampling function can feature program components in the form of one or more instructions for a processor for creating synthetic datasets. The sampling function can be provided for example by it being held in a memory device or loaded into a main memory of a computing unit or generally made available for use.

The sampling function is further embodied in such a way that the synthetic dataset has the same or at least a similar data structure as the underlying real medical dataset. In particular the synthetic dataset takes over at least some of the higher-ranking variables of the medical dataset. Thus if for example the medical dataset has the age, the gender or a medical indication as a higher-ranking variable, then these variables can be contained in the synthetic dataset.

The sampling function is further embodied in such a way that, to create the synthetic dataset, it samples the entire real medical dataset. In other words this means that the sampling function, for creating the synthetic individual datasets, bases them on all original individual datasets - and does not just take account of individual or individual groups of original individual datasets, or just sample specific data classes within the medical dataset. This facilitates the synthetic dataset where possible featuring similar statistical characteristics to the real medical dataset. If individual original individual datasets were not taken into account in the sampling, this would possibly not be guaranteed. The sampling function is further embodied in such a way that it completely replaces the original individual datasets with the synthetic individual datasets.

One idea of forms of embodiment of the present invention is to increase the portability of the information content of medical datasets across organizational boundaries through the creation of a synthetic dataset, the data entries of which can no longer be related back to real existing persons. This enables the synthetic dataset to be exchanged across organizational boundaries without data protection guidelines being violated when this is done. The aforementioned features act synergetically together to the extent that a synthetic dataset can be created, which although it no longer contains any personal data, still extracts the maximum information content from the real existing medical datasets. Thus the information present in the medical datasets such as statistical characteristics, conditional probabilities, data interrelationships is preserved to the greatest possible extent. This enables the synthetic datasets to cover many utilization options that would otherwise only be possible through direct access to the real medical data. The use of a sampling function makes it possible in this case for the medical datasets to be synthesized locally, i.e. within the organization or facility, which possesses the data. The medical datasets thus do not have to be uploaded to a particular location in order to create the synthetic datasets.

Instead the medical datasets can remain stored locally for the entire time.

In accordance with a further form of embodiment a computer-implemented method for utilization of a medical dataset is provided. The medical dataset in this case is stored locally within a first facility. The medical dataset has a number of original individual datasets, which are assigned to real existing patients and have original values for one or more higher-ranking variables. The method comprises a number of steps. One step is directed to the (local) creation of a synthetic dataset through application of the sampling function to the medical dataset, wherein the step of creation is undertaken locally within the first facility, and the synthetic dataset features a number of synthetic individual datasets, which synthetic individual datasets have synthetic values for the same higher-ranking variables as the medical dataset, but cannot be related back to a real existing patient. A further step is directed to the transfer of the synthetic dataset from the first facility to a second facility outside the first facility different from the first facility. A further step is directed to a utilization of the synthetic dataset within the second facility.

The aforementioned explanations, examples, advantages and alternative forms of embodiment also apply to this form of embodiment.

The “first facility” can for example relate to an organization or institution, within which the medical dataset has been obtained and/or is stored. For example the first facility can relate to clinical and/or medical organizations and/or locations and/or entities. Other expressions for “first facility” can be “medical and/or clinical facility” or “local facility”. Such facilities can for example be companies active in the medical and/or clinical sector, medical insurance companies, hospitals, clinics, hospital groups, medical laboratories, practices or similar institutions. The “second facility” can in particular be part of an organization or entity in which information relating to the medical datasets is to be utilized within the framework of clinical and/or medical research, in order for example to develop better diagnosis or treatment methods and corresponding algorithms. The “second facility” can further be part of a health organization such as a hospital for example, in which the information relating to the medical datasets will be utilized within the framework of statistical surveys and evaluations. The “second facility” can in particular relate to a medical technology company, a software company, but also to universities, clinics or groups of clinics conducting research and also to medical insurance companies. Another expression for the “second facility” can in particular be “central unit”.

The first and the second facility can have a data link to one another for exchange of the sampling function and/or of the synthetic dataset. The data link can in particular be wireless or wired. For example the data link can be provided via a network such as the Internet. In particular the second facility can be embodied as a central unit that has a data link to a number of first facilities. In particular the first facility is embodied in such a way that there can be no (direct) access to the medical dataset from outside the first facility.

A utilization of the synthetic dataset within the second facility can basically comprise any evaluation, processing or use. For example a utilization can comprise a training of a trainable classifier to predict a clinical outcome based on the synthetic dataset, and/or a validation of a trainable classifier to predict a clinical outcome based on the synthetic dataset, and/or a statistical evaluation of the synthetic dataset, and/or an archiving of the synthetic dataset in the second facility.

Through the creation of the synthetic dataset it becomes possible, for a utilization, to extract relevant information from a medical dataset and exchange it between different facilities. Since the synthetic datasets are determined in such a way that the content is no longer able to be related back to real existing persons, no data protection policies are violated in such cases. Through the local creation of the synthetic dataset it can moreover be ensured that the medical dataset does not leave the first facility at any time. Through the local creation of a synthetic dataset a large part of the information contained in the medical dataset can still be transported however, whereby the benefit for a utilization can be enhanced. Thus the specified method takes into account the technical and legal circumstances of current data networks in medical technology, which heavily regulate access to the data. The idea of making it possible to exchange data via the creation of synthetic datasets represents a technical solution as to how existing regulations can be complied with and the exchange of information for research and development can still be guaranteed.

In accordance with one form of embodiment the method for utilization of a medical dataset comprises a step of (local) provision of a sampling function within the first facility. The sampling function is embodied in such a way that it creates the synthetic dataset based on the medical dataset.

The “provision of the sampling function” can comprise a downloading of the sampling function to the first facility from a facility different from the first facility. The facility different from the first facility can for example be the second facility or a facility different from the second facility. As an alternative or in addition the “provision of the sampling function” can comprise a loading of the sampling function into a computing unit and/or a main memory of the second facility.

The provision of the sampling function has the advantage that the sampling function does not have to be kept ready by the first facility, but can be made available as required.

In accordance with one form of embodiment the sampling function features a trained function.

A trained function generally maps input data to output data. The output data here can in particular furthermore depend on one or more parameters of the trained function. The one parameter or the number of parameters of the trained function can be determined and/or adapted by training. The determination and/or the adaptation of the parameter or the number of parameters of the trained function can be based in particular on a pair consisting of training input data and associated training output data, wherein the trained function is applied to the training input data for creating training mapping data. In particular the determination and/or the adaptation can be based on a comparison of the training mapping data and the training output data. In general a trainable function, i.e. a function with as yet unadapted parameters, can be referred to as a trained function.

Other terms for trained function are trained mapping specification, mapping specification with trained parameters, function with trained parameters, algorithm based on artificial intelligence, machine-learning algorithm. An example of a trained function is an artificial neural network. Instead of the term “neural network” the term “neural net” can also be used.

A neural network can in particular be trained. In particular the training of a neural network is carried out based on the training input data and the associated training output data in accordance with supervised learning, wherein the known training input data is entered into the neural network and the output data generated by the network is compared with the associated training output data. The artificial neural network learns and adapts the internal parameters independently, for as long as the output data does not sufficiently correspond to the training output data.

One method for supervised learning can appear as follows in accordance with one form of embodiment. To this end a computer-implemented method for provision of a trained function for creating a synthetic dataset based on a medical dataset is provided. The method has the following steps:

provision of training input data, wherein the training input data represents a medical dataset;

provision of training output data, wherein the training output data represents a desired synthetic dataset;

creation of a synthetic dataset by application of the trained function to the training input data;

comparison of the created synthetic dataset with the training output data;

adaptation of the trained function based upon the comparison.

The desired synthetic dataset can for example have been optimized and/or verified by a user with respect to its characteristics.

The use of a trained function as a sampling function has the advantage that the function, as soon as it is sufficiently trained, is able to be applied to many different medical datasets without needing any manual adaptations. Furthermore, such trained functions often deliver better results than algorithms in which a procedure for creating synthetic datasets is predetermined in a fixed manner.

In accordance with one form of embodiment the sampling function has a k-nearest neighbors algorithm.

In principle, k-nearest neighbors algorithms represent a non-parametric method for estimating probability density functions. The inventors have recognized however that such algorithms can be employed for creating synthetic individual datasets with synthetic values. In such cases, for the synthetic values of each synthetic individual dataset, a number of original individual datasets with their corresponding values are taken into account, and indeed those that are “closest” to an original individual dataset singled out - the k-nearest neighbors. The number k in such a case specifies which nearest neighbors are taken into account in each case. Then, from these k-nearest neighbors, a synthetic value is established via a preferably weighted averaging. The number k and/or the weights to be used for the averaging can be predetermined as fixed values, or can be learned via a training method described above or below.

K-nearest neighbor algorithms represent a rapid and flexibly adaptable scheme for sampling real data. By taking into account nearest neighbors synthetic datasets are obtained, which well reflect the (statistical) characteristics of the original medical dataset, but through the weighted average can no longer be related back to real existing persons. A further advantage is that the data structure of the medical dataset is inherently largely preserved and in particular that one or more higher-ranking variables can be transferred automatically.

In accordance with one form of embodiment a number of data classes are defined in the medical dataset and each original individual dataset is assigned a data class. In the step of creation the sampling function is applied separately to each of the data classes, so that for each data class synthetic datasets based on only the original individual datasets assigned to the data class are created.

Data classes are frequently defined in real medical datasets. These can classify for example with regard to a specific illness whether the patient is ill per se or not. The introduction of appropriate data classes can further enable a distinction to be made according to the gender of the patients, their smoking or eating habits, or the age cohort.

The inventors have recognized that the taking into account of such data classes described above in the application of the sampling function enables the membership of a particular class also be to preserved in the synthetic data. Since however all data classes are taken into account “per se”, the data classes of the medical dataset and their statistical characteristics are essentially preserved in the synthetic dataset. A sampling over all these data classes on the other hand would lead to a loss of this information. Thus the benefit of the opportunities to utilize the synthetic datasets would be restricted.

For various utilization scenarios it can even be sensible to define one or more data classes in the medical dataset (in particular if none have yet been set up there). This can be the case for example if a classifier is to be trained and/or validated within the framework of the utilization, which is to classify individual datasets/patients according to membership of a data class. To this end for example an already trained and validated classifier can be downloaded to the first facility (for example by the second facility). This can then be applied to the local medical dataset, whereby one or more data classes can be defined in the medical dataset.

In accordance with one form of embodiment a first data class of the data classes of the medical dataset has a first number of original individual datasets, and a second data class of the data classes, different from the first data class of the medical dataset, has a second number of original individual datasets, wherein the first number is smaller than the second number.

In other words the first data class is thus a minority class of the medical dataset and the second data class of the medical dataset is a majority class. Since there is provision for the sampling function to be applied to all data classes of the medical dataset, there is a synthesizing of both the minority and also the majority class - and not just an “upsampling” of the minority class.

In accordance with one form of embodiment the number of synthetic individual datasets in the synthetic dataset is greater than the number of the original individual datasets in the medical dataset.

Thus in other words, during the synthesizing, the number of individual datasets is simultaneously increased, which for example can improve the data basis for a subsequent utilization. In particular this “upsampling” occurs in this case for all data classes equally, i.e. in a similar or identical ratio of synthetic to original individual datasets. In this way it can be ensured that the class membership and the ratio of the data classes to one another in the synthetic dataset essentially corresponds to the situation on the medical dataset.

In accordance with one form of embodiment the methods further have a step of calculating a quality functional, which quality functional is defined as a measure for the match between the statistical characteristics of the synthetic dataset and the statistical characteristics of the original dataset.

Through the calculation of the quality functional an objective criterion is provided as to how well or badly the information content of the medical dataset possibly relevant for a later utilization can be transferred to the synthetic dataset. In other words the quality functional is construed as a measure or how realistic or close to reality the synthetic data is. The quality functional can in particular be created within the first facility (locally). The quality functional can be transferred to the second facility from the first facility in particular together with the synthetic dataset.

In accordance with one form of embodiment at least one parameter of the sampling function is optimized by optimizing the quality functional for the medical dataset.

In accordance with one form of embodiment the optimization in this case in particular comprises the steps:

definition of a number of selection values for the parameter;

creation of a synthetic dataset in each case for each of the number of selection values, wherein the respective selection value is used as a value for the parameter of the sampling function to be optimized;

computation of the quality functional for each synthetic dataset created,

comparison of the computed quality functionals;

selection of an optimal selection value for the parameter to be optimized based on the comparison.

The optimization of the quality functional can in particular comprise a (local) extreme value of the quality functional being identified as a function of the one or more parameters of the sampling function to be optimized, to which (local) extreme value the statistical characteristics of the synthetic dataset and of the medical dataset are well matched. A good match in this respect can in its turn increase the value of the synthetic dataset for a subsequent utilization. Using the example of a k-nearest neighbor algorithm, a parameter to be optimized can for example be the number k of the nearest neighbors, which are taken into account during sampling for creating a synthetic value/individual dataset. The optimization of the sampling function takes place in particular within the first facility. Thus the sampling function can be matched specifically to the respective medical dataset, without the dataset having to leave the first facility. The optimization described here can also be referred to as (in particular unsupervised or semi-supervised) training of the sampling function.

In accordance with one form of embodiment the methods further have the step of selecting variables to be sampled from the higher-ranking variables, wherein in the creation step the sampling function is only applied to those original values of the medical dataset that belong to the selected variables to be sampled, so that the synthetic dataset merely has synthetic values for the variables to be sampled.

The background to this is that the medical dataset under some circumstances contains variables that are not only not relevant for the utilization, but are also problematic for data protection legislation reasons. Furthermore, individual higher-ranking variables can falsify the statistical characteristics of the synthetic dataset. Through the choice of variables to be sampled or the deselection of higher-ranking variables that are not to be sampled, these problems can be taken into account. Thus a synthetic dataset can be provided which not only reproduces the medical dataset well, but can also guarantees that personal data is highly secure.

The selection of variables to be sampled can take place automatically in accordance with forms of embodiment. For example the higher-ranking variables of the medical dataset can be reconciled automatically with a blacklist or whitelist. The blacklist in this case can feature higher-ranking variables that are not to be sampled (i.e. associated original values of the medical dataset to which the sampling function is not be applied, so that the synthetic dataset does not feature the higher-ranking variables contained in the blacklist). An example of variables contained in the blacklist can be a name of a doctor for example. Conversely those higher-ranking variables that are to be sampled can be contained in the whitelist (i.e. to which the sampling function is to be applied, so that the synthetic dataset merely features synthetic values for the variables contained in the whitelist). The blacklist or whitelist can be created by a user for example and/or be adapted to the utilization and/or to existing data protection regulations. Furthermore the variables to be sampled can be selected manually by a user. To this end a user interface can be provided within the first or second facility. In addition or as an alternative a semi-automatic selection of the variables to be sampled is possible, in which (e.g. via a suitable user interface within the first and/or second facility) variables to be sampled for the medical dataset are automatically (e.g. based on whitelists and/or backlists) proposed to a user, which the user can then supplement, process and/or confirm.

In accordance with one form of embodiment one of the higher-ranking variables of the medical dataset refers to an absolute point in time at which the original values of an original individual dataset were recorded. In other words the medical dataset thus also features longitudinal data. The methods can then further have a step of converting the absolute points in time into relative time intervals, wherein the relative time intervals are each defined within groups of the original individual datasets, which groups are defined by the assignment of the original individual datasets to the same patient, and the earliest absolute point in time within a group is used as the reference time for computing the relative time intervals.

In other words a reference point in time is defined for each patient. This can then serve as “time zero” for all subsequent points in time. Thus account can be taken of the fact that, although the relative time intervals between individual examinations can be relevant for assessment and utilization of medical data, in order for example to assess the progression of a condition, the absolute point in time however is often of less importance. What is more, the sampling via an absolute point in time in the medical context can often lead to systematic errors being induced in the synthetic dataset, since similar absolute points in time could suggest similarities between individual datasets, which would not be justified only based upon the medical indication. By the conversion of the absolute points in time into relative time intervals, against this background a scheme is implemented that eliminates possible sources of error, which works out medically relevant information and furthermore makes possible an automated creation of the synthetic dataset.

In accordance with one form of embodiment, in the creation step for the creation of a synthetic individual dataset, only the original individual datasets are sampled that belong to the same patient.

In other words in this way virtual or synthetic patients are set up in the synthetic dataset. The synthetic individual datasets of a synthetic patient in this case go back to the original individual datasets of a real patient. In this way conditional probabilities, which stem from the assignment of the original individual data to specific patients, will be contained in the synthetic dataset. However this type of patient specificity is not absolutely necessary for all applications/utilizations. Therefore as an alternative there can also be provision to sample across all original individual datasets independent of their assignment to a patient.

In accordance with a further embodiment a system for utilization of a medical dataset is provided. In this case the medical dataset is stored locally in a facility (or organization). The medical dataset features a number of original individual datasets, which are assigned to real existing patients and have original values for one or more higher-ranking variables. The system has a computing unit, which is arranged outside the facility. The system further has an interface for communication between the computing unit and the first facility. The computing unit is embodied to induce a local creation of a synthetic dataset in the facility via the interface, so that the synthetic dataset has a number of synthetic individual datasets, which have synthetic values for the same higher-ranking variables as the medical dataset, but cannot be related back to real existing patients. The computing unit is further embodied to receive the synthetic dataset from the facility via the interface and to utilize the synthetic dataset outside the facility.

In this case the “facility” can correspond to the aforethe “first facility”, and in particular can be embodied as a local (medical) facility or organization.

The computing unit can be embodied as a central or decentral computing unit. The computing unit can have one or more processors. The processors can be embodied as a Central Processing Unit (abbreviated to CPU) and/or as a Graphics Processing Unit (abbreviated to GPU) and/or in the form of other computing modules such as Tensor Processing Units (TPUs). As an alternative the computing unit can be implemented as a local or cloud-based processing server.

The interface can be embodied in general for exchange of data between the computing unit and the facility. The interface can be implemented in the form of one or more individual data interfaces, which can have a hardware and/or software interface, e.g. a PCI bus, a USB interface, a Firewire interface, a ZigBee or a Bluetooth interface. The interface can further feature an interface of a communication network, wherein the communication network can feature a Local Area Network (LAN), for example an Intranet or a Wide Area Network (WAN). Accordingly the one or more data interfaces can have a LAN interface or a Wireless LAN interface (WLAN or Wi-Fi).

The advantages of the proposed system can essentially correspond to the advantages of the proposed methods. Features, advantages or alternate forms of embodiment can likewise be transferred to the other claimed subject matter and vice versa.

The inducing of the local creation can for example comprise the computing unit of the facility providing a sampling function locally, which is embodied, based on the medical dataset, to create the synthetic dataset. The local provision in this case can comprise a download of the sampling function to the facility. The inducing can further comprise the computing unit controlling an application of the sampling function to the medical dataset.

The system can in particular be embodied in such a way that a direct access of the computing unit to the medical dataset stored locally in the facility is not possible. This can be guaranteed for example by a corresponding protection of the data memory within the facility from external access.

The invention further relates, in a further embodiment, to a computer program product, which comprises a program and is able to be loaded directly into a memory of a programmable computing unit and has program means, e.g. libraries and auxiliary functions, for carrying out a method of an embodiment for creating a synthetic dataset for cross-facility utilization of medical datasets, when the computer program product is executed.

The computer program products in this case can comprise software with a source code that still has to be compiled and linked or only has to be interpreted, or an executable software code, which for execution only has to be loaded into a processing unit. The processing unit in this case can comprise the aforethe computing unit and/or local computing units within the aforethe local facilities (i.e. the “first facility” or the “facility”). The computer program products enable the methods to be carried out in a rapid, identically repeatable and robust manner. The computer program products are configured so that the processing units can carry out an embodiment of the inventive method steps. The processing unit in such cases must have the respective prerequisites such as for example a corresponding main memory, a corresponding processor, a corresponding graphics card or a corresponding logic unit, so that the respective method steps can be carried out efficiently.

The computer program products are stored for example on a computer-readable storage medium or are held on a network or server, from where they can be loaded into the processor of the respective computing unit, which can be directly connected to the processing unit or can be embodied as a part of the processing unit. Furthermore control information of the computer program products can be stored on a computer-readable storage medium. The control information of the computer-readable storage medium can be embodied in such a way that, when the data medium is used in a computing unit, it carries out an inventive method. Examples of a computer-readable storage medium are a DVD, a magnetic tape or a USB stick, on which electronically-readable control information, in particular software, is stored. When this control information is read from the data medium and stored in a computing unit, all forms of embodiment of the method described above can be carried out. In this way the invention can also be based on the the computer-readable medium and/or the the computer-readable storage medium. The advantages of the proposed computer program products or of the associated computer-readable media essentially correspond to the advantages of the proposed methods.

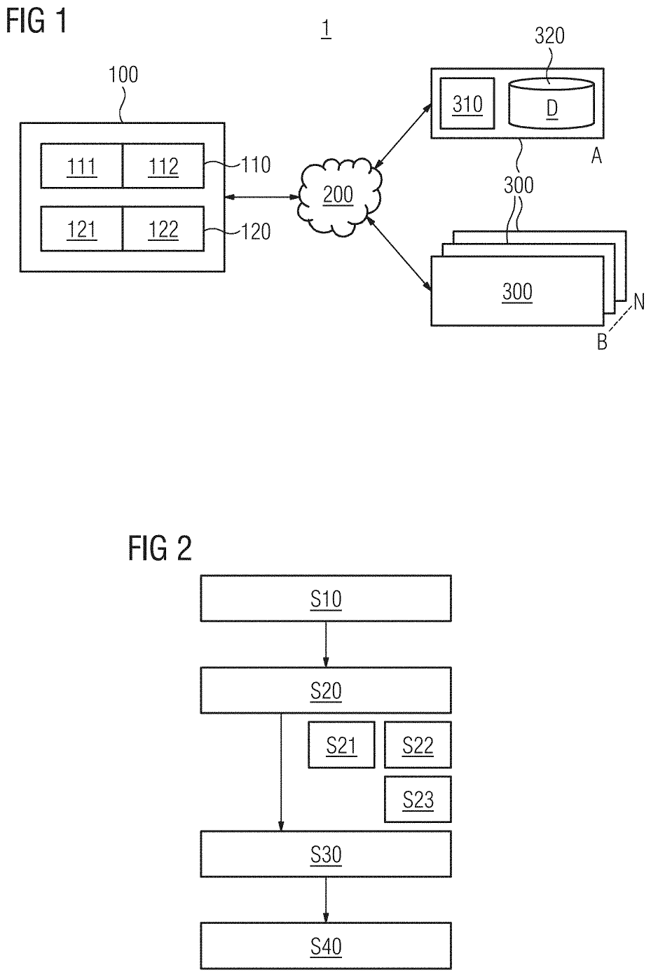

FIG. 1 shows an example embodiment of a system 1 for utilization of a medical dataset MD, which is stored locally in a local facility A (or local organization). The system 1 is embodied to carry out embodiments of the methods described in more detail in conjunction with FIGS. 2 to 7.

The system 1 has a central unit 100 and one or more local clients 300. The local clients 300 are each assigned to different local facilities A . . . N. The central unit 100 and the local clients 300 are connected via a network 200.

The central unit 100 is generally embodied to initiate, to coordinate and to control the utilization of the medical data MD. The utilization of the medical data MD can generally comprise an evaluation of the medical data MD, wherein the data processing steps for utilization of the medical data MD occur in the central unit 100. The local facilities A . . . N can for example relate to clinical or medical environments and/or organizations and/or sites and/or facilities. These can be companies, health insurance companies, hospitals, clinics, hospital groups, medical laboratories, practices or similar institutions for example.

The utilization of the medical data MD can in particular comprise the storage and evaluation of information which is derived from the medical data MD. The utilization can further comprise a training of a trainable function or its validation based on the medical data MD. The trainable or trained functions can generally carry out tasks, which would otherwise usually need human intellectual activity. In such case the trainable or trained functions imitate cognitive processes of the human intellectual activity. Within the system 1 such tasks can comprise the creation of medical diagnoses and/or prognoses, the identification of lesions in medical image data, the annotation of medical data, the creation of medical findings and the like. In particular the trainable or the trained function in such cases can have an electronic classifier function (“classifier” in the following), which is embodied to assign individual datasets of a medical dataset MD to one or more classes (e.g. whether an individual dataset or a group of individual datasets indicate a specific illness or not).

The central unit 100 can be a web server for example. The central unit 100 can further be a cloud server or a local server. The central unit 100 can be implemented by any types of suitable computing facility. The central unit 100 can have a computing unit 110 and a memory unit 120.

The computing unit 110 can have one or more processors and a main memory. The one or more processors can be embodied for example in the form of one or more Central Processing Units (CPUs), Graphics Processors (GPUs) and/or other computing modules such as Tensor Processing Units (TPUs). The computing unit 110 can further have a microcontroller or an integrated circuit. As an alternative the computing unit 110 can have a real or virtual group of computers in the form of a cluster or a cloud. The main memory can have one or more computer-readable memory media such as a RAM for temporary loading of data. This data can for example be data from the memory unit 120 or data that has been uploaded from local clients 300. The main memory can further store information in such a way that the information is accessible to the one or more processors. This information can comprise instructions, which can be executed by the one or more processors. These instructions can comprise instructions for uploading a sampling function DSF to the local clients 300, instructions for carrying out the sampling function DSF at the local clients 300, for receiving the synthetic data SD created by the sampling function DSF from the local clients 300 and for utilizing the same in the computing unit 110.

The sampling function DSF in this case is a function or an algorithm that is generally embodied to create a synthetic dataset SD based on an original dataset MD. The sampling function DSF is embodied to create the synthetic dataset SD so that the dataset has the same data structure and where possible similar statistical characteristics to the original dataset MD. In this case all original higher-ranking variables can be taken over from the original dataset MD or just a part thereof. In other words the higher-ranking variables of the synthetic dataset SD where possible have a correspondence in the original dataset MD. The sampling function DSF is further embodied to create the synthetic dataset SD in such a way that the information contained therein does not allow any reference back to individual instances of data in the real dataset MD.

In particular the sampling function DSF can likewise have a trained function, which is embodied (trained) in such a way that, based upon an original dataset MD, it creates a synthetic dataset SD with the described characteristics. Further details in respect of the sampling function DSF are specified further below in conjunction with FIGS. 4 to 7.

The central unit 100 can be implemented using a server facility or using a number of server facilities. If a number of server facilities are used, these can operate in a parallel or serial arrangement or in a combination of the two. The central unit 100 can further have an interface unit (not shown), which is embodied for communication with the local clients 300 via the network 200. The interface unit can have any given components that are suitable for establishing a connection for one or more networks. These components can be embodied for example as transmitters, receivers, ports, controllers or antennas.

The memory unit 120 can be embodied as cloud memory. As an alternative the memory unit 120 can be embodied as local memory with one or more elements within the central unit 100. The memory unit 120 can have one or more memory modules. A number of databases can be set up in the memory unit 120. One of these databases can be embodied as a tool database 121, which is embodied in particular to store and/or to keep in reserve the sampling function DSF and/or one or more trained or trainable functions for utilization of the medical data MD.

The trainable or trained functions can include modules that are primarily embodied for classification of datasets and/or for derivation of numerical forecasts and/or for clustering of datasets. In particular the functions can feature one or more neural networks (e.g. so-called Deep Neural Networks, Recurrent Neural Networks, Convolutional Neural Networks, Convolutional Deep Neural Networks, Adversarial Networks, Deep Adversarial Networks and/or Generative Adversarial Networks etc.) or be based on these. The functions can furthermore feature the functions Bayesian networks, Decision Trees, Random Forest Module, linear or logistical regression model, k-means clustering module, Q-learning module and/or genetic algorithms or be based on these.

A further database within the memory unit 120 can be embodied as a data memory 122 for storing synthetic data SD that has been created by the sampling function DSF based upon the local medical datasets MD and has been uploaded from the local facilities A . . . N into the central unit 100.

The computing unit 110 can have modules 111 and 112, in order to control the utilization of the original dataset MD. In this case the module 111 can be construed as a data provision module, which is embodied to extract synthetic data SD from the original dataset MD—without the original dataset MD leaving the respective local site. For this the data provision module 111 can be embodied to download the sampling function DSF to the respective client 300 and/or to apply the sampling function DSF to the local data MD of the client 300 or to initiate its application. The data provision module 111 can further be embodied to initiate an upload of the synthetic data SD created locally by the sampling function DSF from the client 300 to the central unit 100 and/or to receive the uploaded synthetic datasets SD.

Module 112 can be construed as a data utilization module. The data utilization module 112 can be embodied to load a trained function from the tool database 121 and apply it to one or more synthetic datasets SD, in order in this way for example to train a trainable function. As an alternative or in addition the data utilization module 112 can be embodied to validate a trained function based on one or more synthetic datasets SD. What is more the data utilization module 112 can be embodied to evaluate one or more synthetic datasets statistically. The data utilization module 112 can further be embodied to archive one or more synthetic datasets SD in the data memory 122 for a later utilization.

The division into modules 111 and 112 serves as explanation and is to be understood as being by way of example and not as restrictive. Accordingly the modules 111 and 112 can also be integrated into a processing unit or embodied in the form of (computer) program sections, which are embodied to carry out the corresponding method steps.

The central unit 100 can exchange information with one or more local clients 300 over the network 200. In this case any number of local clients 300 can be connected to the central unit 100 over the network 200.

The local clients 300 can have a client computing unit 310 and a client memory unit 320. The client memory unit 320 can be embodied as a local memory unit within the local client 300. In particular the client memory unit 320 can have one or more local databases, in which the respective local datasets MD of the local facilities A . . . N are stored.

The local datasets MD form the original datasets of which the information content is to be utilized in the central unit 100. In particular the local datasets MD are medical datasets, which have personal data for one or more patients. The medical datasets can in each case have a number of individual datasets, which are each assigned to patients of the respective local facility A . . . N. These individual datasets are referred to below as original or real individual datasets. In such cases a number of different original individual datasets can also be assigned to a patient, which can for example relate to examinations of the patient at different times. Each original individual dataset has original values for one or more higher-ranking variables. The original values can for example have laboratory values, vital values, examination parameters (e.g. number of painful joints), personal information about patients, information about the patient's medication etc.. The original values can further relate to variables and/or features that have been extracted from text or image data. Text data in such cases can be pathological and/or radiological findings. Image data can in particular be medical image data (e.g. radiology or histopathology images). Higher-ranking variables then define a type of category of the original values. One or more higher-ranking variables of an original dataset MD can be numeric variables, which relate to numeric original values. These can for example comprise an ID, an age, the time at which the individual dataset was obtained, one or more inflammation parameters or the blood pressure of a patient. As well as this one or more higher-ranking variables can be categorical variables, which relate to non-numeric values. Such non-numerical values can for example contain simple binary expressions such as ‘yes’ or ‘no’, or classifications such as ‘low’, ‘medium’, or ‘high’. As well as this they can feature names, of a medication for example, or free texts such as a finding of a doctor for example. The original individual datasets in such cases can address higher-ranking variables differing from one another. The local datasets MD are created at the respective local facility A . . . N and/or managed by the respective local client 300. The local datasets MD can be provided for example as Electronic Medical Records (EMR).

As already stated, the local datasets MD are preferably stored locally within the local clients 300 in one or more local databases 320. These databases can for example be part of a Hospital Information system (HIS), Radiology Information system (RIS), Laboratory Information System (LIS), Cardiovascular Information System (CVIS) and/or Picture Archiving and Communicating System (PACS). The local datasets MD can be retrieved from these databases 320 and entered into the sampling function DSF for example. The local data access and the application of the sampling function DSF to the local datasets MD can be controlled by the client computing unit 310. According to a few forms of embodiment the local datasets MD are only accessible, because of data protection regulations or other restrictions, within the respective local client 300/the local facility A . . . N. The local clients 300 are in particular embodied in such a way that there cannot be any access from outside the local facilities A . . . N to the local datasets MD. The client memory unit 320 can be continuously updated with new examination or test results (e.g. continuously or regularly, such as e.g. daily, weekly, etc.).

The client computing units 310 can have any suitable type of computing facility, e.g. a PC or laptop, a local server or a local server system. The client computing units 310 can have one or more processors and a memory. The one or more processors can be designed for example in the form of one or more Central Processing Units (CPUs), Graphics Processing Units (GPUs), and/or other computing systems. The memory can have one or more computer-readable media and store commands for the processor. The commands can in particular comprise instructions for applying the sampling function DSF to a local dataset MD, in order to create a synthetic dataset SD.

Like the central unit 100, the local clients 300 can have an interface (not shown), in order to make the connection via the network 200 to the central unit 100 for example and exchange data. The interface can have any components suitable for this task, such as e.g. transmitters, receivers, ports, controllers or antennas.

The network 200 can feature any communication network, such as e.g. a local network in the form of an intranet or a Wide Area Network, such as the Internet. The network 200 can further feature a mobile radio network, or a wireless network as well as a combination of the aforementioned components. In general communication over the network can be via wireless or wired network interfaces using various communication protocols (e.g. TCP/IP, HTTP, SMTP, FTP) or formats (e.g. HTML) and/or secure connections (e.g. VPN, HTTPS, SSL).

Methods in accordance with forms of embodiment of the invention with regard to FIGS. 2 to 7 will be explained below. The flow diagrams shown serve equally as examples for hardware-based circuits or machine-readable commands for implementing the method steps in the form of one or more computer program products. Computer program products can be embodied in the form of software, which is stored on non-volatile, machine-readable memory media, such as for example on a CD-ROM, a floppy diskette, a hard disk, a DVD or memory assigned to the processor (main memory). As an alternative, the methods or parts thereof can be carried out by facilities other than processors or can be caused to execute in the form of firmware or hardware components. Although example methods are described with regard to FIGS. 2 to 7, other method-type forms of embodiment can also be derived from the disclosure given below. For example the sequence of the individual method steps can vary or individual steps can be exchanged or left out. Moreover method steps shown as separate can be combined.

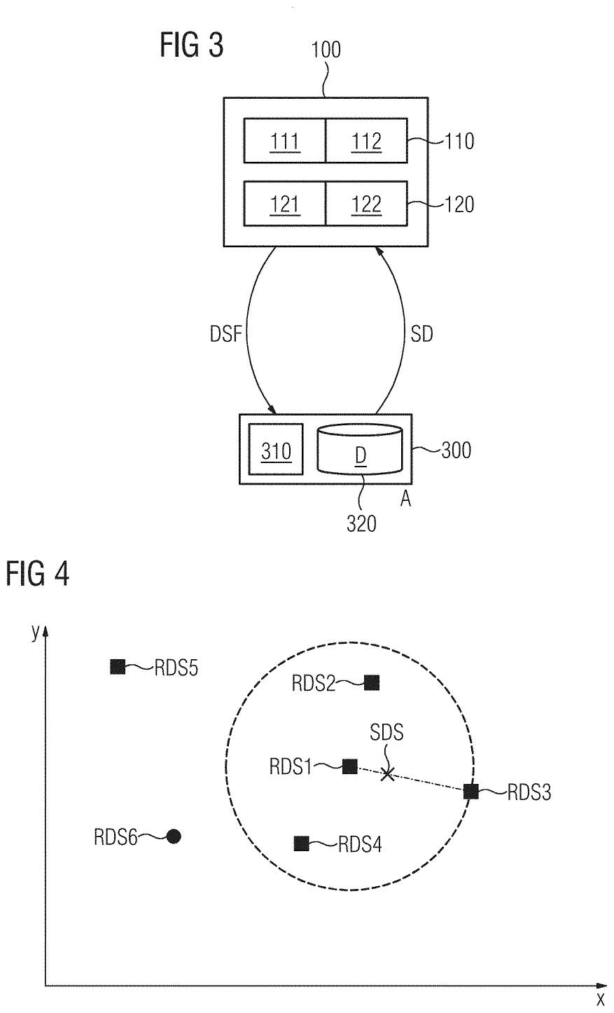

FIG. 2 shows a flow diagram for a method for utilization of a medical dataset MD in accordance with one form of embodiment. Data flows between the components of the system 1 associated therewith are shown in FIG. 3.

A first step S10 is directed to the (local) provision of the medical dataset MD stored locally in the client 300. In this case the client computing unit 310 can be embodied to access the client memory unit 320 and load the medical dataset MD.

In a next step S20, based on the medical dataset MD, a synthetic dataset SD is created. The creation of the synthetic dataset SD is undertaken in this case by the real values of the medical dataset MD being replaced, so that the information contained in the synthetic dataset SD cannot be related back to the real persons (patients). Technically the creation of the synthetic dataset SD can be brought about with the aid of a sampling function DSF. The sampling function DSF is embodied to sample the medical dataset in order to create the synthetic dataset SD. The application of the sampling function DSF can be undertaken in optional substeps S21-S23 of step S20. A substep S21 is directed to the transfer of the sampling function DSF from the central unit 100 to the local client 300. If the sampling function DSF is already present on the local client 300, substep S21 can be omitted. This can be the case for example when the sampling function DSF is still present within the local client 300 from an earlier execution of the method, or the sampling function DSF has been downloaded to the local client 300 from a third facility (i.e. not the central unit 100). In substep S22 the sampling function DSF is loaded by the client computing unit 310. In a further substep S23 there is the application of the sampling function DSF to the medical data MD. To this end the medical data MD is entered into the sampling function DSF. Then, as output of the sampling function DSF, the synthetic data SD is obtained. Details in respect of the data sampling of step S20 and the way in which the sampling function DSF functions are explained further below with regard to FIGS. 4 and 5. In order to ensure that the medical data does not leave the local facility A, step S20 and also where necessary the substeps S22 and S23 take place locally, i.e. in the local client 300.

In step S30 the synthetic dataset SD is transferred to the central unit 100, where it is subsequently processed (utilized) in step S40. The step S30 in this case can occur automatically or only be enabled after a further monitoring by the respective local facility A . . . N. During the monitoring, a check can be made as to whether the information in the synthetic dataset SD has been sufficiently anonymized or pseudonymized, so that the information contained therein cannot be related back to actually existing persons. This monitoring can be carried out automatically, but also by a user. Since the synthetic dataset SD represents a sample of the medical dataset MD, which indeed preserves its fundamental data structure and the statistical characteristics (the latter at least essentially), but excludes an assignment to real existing patients, in this way the information contained in the medical dataset MD can be utilized without violating data protection guidelines or the like.

The utilization in step S40 can have different aspects. For example one or more trainable functions can be trained in step S40 based on one or more synthetic datasets SD, in order then to use these for comparable medical datasets MD. Furthermore one or more of the synthetic datasets SD created in this way can be used for validating an existing trained function. Since the statistical characteristics of the underlying medical dataset MD are preserved during the sampling by the sampling function DSF, a utilization can further consist of a statistical evaluation, which e.g. makes possible a statement about macroscopic state variables of the patient population underlying the medical dataset MD. Finally a utilization can also consist of an archiving of the synthetic dataset SD in the memory unit 122, if necessary for later use. During archiving, metadata relating to the underlying medical dataset MD or the creation process of the synthetic dataset SD can be stored together with the synthetic dataset SD. Such metadata can feature a data dictionary (i.e. a description of the individual higher-ranking variables, of their units and intervals), the number of the individual datasets in the medical dataset MD, performance logs of the creation process by the sampling function DSF and/or one or more metrics, which describe the quality of the synthetic dataset SD. The metadata can be transferred to the central unit 100 in this case in step S30 from the local clients 300 together with the synthetic dataset SD. Metrics, which specify the quality of the synthetic dataset SD, can for example be functions that quantify the extent to which statistical characteristics of the synthetic dataset SD match those of the original dataset MD. Such metrics can also be referred to below as quality functional(s).

Forms of embodiment of the sampling function DSF will now be described with regard to FIG. 4. Basically the sampling function DSF is a function that is embodied in such a way that it samples an original, in particular medical, dataset MD in order to create a synthetic dataset SD. In this case the original individual values contained in the original dataset MD are processed in order to create synthetic values. The processing of the original values in this case follows a scheme that is designed where possible to preserve the data structure of the original dataset MD, i.e. in particular the type and number of higher-ranking variables from the original dataset MD, provided this is sensible against the background of a subsequent utilization. The sampling function DSF is further embodied to transfer the statistical characteristics of the original dataset MD where possible to the synthetic dataset SD, so that the synthetic dataset SD where possible has similar statistical characteristics to the underlying original dataset MD.

In particular the sampling function DSF can have one or more trained and/or trainable functions and/or function components in order to implement these requirements. A trained function in this case in quite general terms maps input data to output data. The output data here can in particular furthermore depend on one or more parameters of the trained function. The one or more parameters of the trained function can be determined and/or adapted by training. The determination and/or the adaptation of the one or more parameters of the trained function can be based in particular on a pair consisting of training input data and associated training output data, wherein the trained function is applied to the training input data for creating training mapping data. In particular the determination and/or the adaptation are based on a comparison of the training mapping data and the training output data. In general a trainable function, i.e. a function with one or more parameters not yet adapted, can also be referred to as a trained function. In the present case training input data can be formed for example by a medical dataset. The training output data can then be a predetermined associated synthetic dataset SD that has the desired characteristics. Other terms for trained function are trained mapping specification, mapping specification with trained parameters, function with trained parameters, algorithm based on artificial intelligence, machine learning algorithm.

An example of trainable functions that are suitable for the requirements on the sampling function DSF are k-nearest neighbors algorithms, of which the operating principle is shown in FIG. 4. For the sake of simplicity and without restricting the generality, the diagram in FIG. 4 is restricted to two higher-ranking variables x and y. The sampling function DSF is however naturally able to be applied for datasets with any given number of dimensions.

Each data point RDS1, RDS2, RDS3, RDS4, RDS5, RDS6 in the coordinate system spanned by the variables x and y can be construed as an original individual dataset of the original dataset MD. Each original individual dataset then has as its original values the x- and y-values of the respective data point RDS1, RDS2, RDS3, RDS4, RDS5, RDS6. In order to determine a synthetic individual dataset SDS based on the original individual datasets RDS1, RDS2, RDS3, RDS4, RDS5, RDS6, first of all a data point RDS1 (or original individual dataset) is selected. For this the k-nearest neighbors are then determined (RDS2, RDS3, RDS4). In the example shown k=3. k in this case is a parameter of the algorithm, which can be adapted to the respective original dataset MD (e.g. by way of an optimization). As the next step one of the k-nearest neighbors of the selected data point RDS1 is selected at random (here: RDS3). Next a new synthetic data point SDS (or synthetic individual dataset) somewhere on the distance vector between the dataset RDS1 considered at that moment and the selected nearest neighbor RDS3 is determined at random. This can be construed by the formula

SDS=RDS1+μ(RDS3−RDS1),

in which μ is a random number from the interval [0,1].

The original individual datasets RDS1, RDS2, RDS3, RDS4, RDS5, RDS6 of the original dataset MD can further be assigned to one or more classes. With a medical dataset MD the classes can for example be defined as an illness label (healthy vs. ill). The class with the lowest number of individual datasets in this case is referred to as the minority class. The further classes then form one or more majority classes of the dataset MD. In order to preserve the class membership when sampling the original dataset MD, this can be taken into account in the selection of the nearest neighbors. In other words, in the selection of the nearest neighbors only those original individual datasets are selected which belong to the same class. This is indicated in FIG. 4 by round or square symbols of the data points. All data points with round points belong to one class, while all data points with square symbols belong to another class.

In order to optimize the creation of the synthetic datasets SD, and in particular to adapt them to the original dataset MD, various parameters of the sampling function DSF can be optimized. Parameters that are optimized during the creation of the synthetic data can in particular be hyperparameters of the sampling function DSF. Such hyperparameters can refer to “higher-ranking” parameters of the sampling function DSF, which determine the fundamental behavior and where necessary the sequence of the training of the (trained) sampling function DSF. For a k-nearest neighbors algorithm this is above all the number k of the nearest neighbors. The ratio of the synthetic individual datasets between the classes of the synthetic dataset SD or the total number of synthetic individual datasets in the synthetic dataset SD can be further optimized.

The creation of synthetic data can also be employed for “upsampling” underrepresented data classes. This enables the class distribution of a dataset to be adapted. The class distribution in this case can refer to the relationship of the number of individual datasets between the different classes in a dataset. Above all in medical datasets MD it can occur that a class is underrepresented in relation to one or more classes. This class is also referred to as a minority class. When training a trainable function, which for example is to recognize membership of the minority class, it can occur that the minority class contains too few individual datasets for a sensible training. In other words a trainable function is then “biased” by the majority class(es). If for example a classifier is to be trained, which is to decode based upon the patient data whether a patient is ill, there must be sufficient verified cases of ill patients present in the training phase of the classifier. This is frequently not guaranteed with medical datasets MD. This is all the more problematic since error classifications of the minority class, i.e. “false negative” cases, can have serious consequences precisely with medical applications. The creation of “new” instances in the minority class can provide assistance here.

K-nearest neighbors algorithms can in particular be employed for this, which are then applied selectively and exclusively to the minority class to be “upsampled”. Examples of this are SMOTE (Synthetic Minority Over-sampling Technique) or ADASYN (Adaptive Synthetic Sampling Approach for Imbalanced Learning). It is an idea of forms of embodiment of the present invention to employ algorithms for sampling the minority class, such as e.g. SMOTE or ADASYN and their variants, in the creation of a complete synthetic dataset SD. Deviating from the original conception of these algorithms however, in this case it is not only new individual datasets that are created within a selected class of the dataset. Instead all classes or individual datasets of the original dataset MD are taken into account in order to create synthetic individual datasets. Thus all classes of the original dataset MD are sampled or upsampled. What is more, in the present case the original individual datasets of the original dataset MD are not transferred into the synthetic dataset SD, but are replaced completely - in order to satisfy any data protection policies or the like. The number of the synthetic individual datasets can in this case in particular be greater than the corresponding number of individual datasets in the underlying medical dataset MD. As a further difference the distribution ratio of the synthetic individual datasets to the data classes corresponds where possible to the distribution ratio of the original individual datasets to the data classes of the underlying original dataset MD. Thus, unlike upsampling, only one class will preserve the prevalence of the individual data classes. In order to realize this there can in particular be provision for sampling each data class separately, i.e. for only using original individual data of the same class in the computation of synthetic individual data of a class.

The basic execution sequence of the creation of a synthetic dataset SD based upon an original dataset MD using the sampling function DSF is shown in FIG. 5.

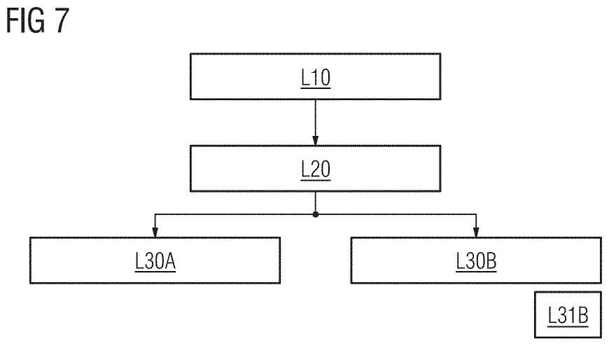

A first step D10 is directed to the preparation of the original dataset MD. This step can serve to adapt the format of the original dataset MD (which, depending on local facility A . . . N, can have marked anomalies) to the requirements of the sampling function DSF. Step D10 can further include normalizing or standardizing the original values contained in the original individual datasets, especially for different scales of the values of individual variables (optional substep D11). At least two classes can further be defined in the original dataset MD (optional substep D12). The definition of the classes can be required for the use of some sampling functions DSF (such as e.g. SMOTE) or already occur in respect of a later utilization of the synthetic dataset SD. If, based upon synthetic data SD, a classifier for recognition of symptoms is to be trained in the central unit 100 later for example, it can be sensible to use the values ‘ill’ or ‘healthy’ in the original dataset MD for definition of corresponding classes (and not as original values to be sampled). If such information is not yet set up in the original dataset MD, it can be created locally in step D12 by local application of a trained classifier to the original dataset MD for the respective client 300. Further, in an optional substep D13, longitudinal data can be recognized and prepared accordingly for further processing by the sampling function DSF. Longitudinal data in this case is series or groups of individual datasets that are assigned to one and the same patient, but have been recorded at different points in time. In other words longitudinal data maps the temporal development of one or more original values of a patient. Further details for handling longitudinal data will be given below in conjunction with FIG. 7. In a further optional substep D14 one or more higher-ranking variables can be selected from the higher-ranking variables of the medical dataset MD, which are to be taken into account in the creation of the synthetic dataset. The synthetic dataset SD then only has values for the selected higher-ranking variables. The background is that as a rule not all original values in the original dataset MD are relevant for the utilization of the information contained therein. Thus for example the name of the doctor undertaking the treatment can be irrelevant, when a classifier for recognition of illness is to be trained using patient data as its starting point. More than that, some variables can be problematic in respect of the use of the sampling function DSF. Thus for example the doctor's name can suggest a similarity in the search for nearest neighbors, which is not justified only based upon the actual relevant higher-ranking variables. The selection of the higher-ranking variables in substep D14 can take place automatically or manually by a user, who can enter their choice for example via a user interface in the central unit 100.

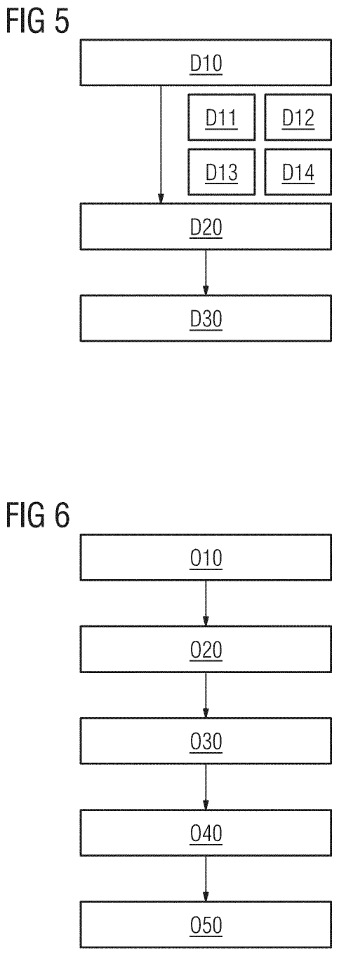

The next step D20 in FIG. 5 is then directed to an optimization of the sampling function DSF. The aim of the optimization of the sampling function DSF in step D20 can be to adapt the sampling function DSF to the specific circumstances on the local client 300 to the local facility A. In particular in this case there can be an adaptation of one or more (hyper)parameters of the sampling function DSF to the original dataset MD. In this way, with a use of k-nearest neighbor algorithms, it can occur for example that for different original datasets MD different k-values are also optimal. Further details in respect of the optimization of the sampling function DSF are specified below with regard to FIG. 6. The step D20 is to be understood as optional in this case and can also be omitted, if e.g. a pre-configured sampling function DSF already delivers acceptable results.

In step D30 the sampling function DSF is finally applied to the original dataset MD. In other words the original dataset MD is entered into the sampling function DSF. By application of the sampling function DSF to the original dataset MD a synthetic dataset SD is created based on the original dataset MD. In this case all individual datasets of the original dataset MD are preferably taken into account, i.e. the original dataset MD is completely sampled. In particular the sampling of the original dataset MD is thus not restricted to individual classes, but relates to all classes of the original dataset MD. If the original dataset MD contains a minority class and a majority class for example, the synthetic dataset SD is created by both the original individual datasets of the minority class and also the original individual datasets of the majority class being sampled (and replaced). The classes in this case are preferably sampled independently of one another, i.e. without overlapping of the individual datasets of different classes.

In step D20, as mentioned, there can be an optimization or adaptation of the sampling function DSF to the respective original datasets MD. Described below are associated examples of method steps with regard to FIG. 6. For the sake of simplicity it is assumed below that only one parameter of the sampling function DSF is to be optimized. The method is however similarly applicable for optimizations in a multi-dimensional parameter space.

First of all, in step O10, for the parameter of the sampling function DSF to be optimized, a number of possible selection values are defined. Using the example of a k-nearest neighbors algorithm, the parameter to be optimized can be the number k of the nearest neighbors. This can for example amount to 1, 2, 3, 4 etc.. These numbers in this example then represent the possible selection values for the parameter k to be optimized.

In step O20 a synthetic dataset SD is then created for each selection value. For each selection value the sampling function DSF is applied for this to the original dataset MD. Since the sampling of the original dataset MD with the sampling function DSF depends on the parameter to be optimized, the synthetic datasets SD created in this way will be different and have different characteristics by comparison with the original dataset MD.

In order to decide which of these synthetic datasets SD best corresponds to the characteristics of the original dataset MD (and thus: which selection parameter is best suited to the original dataset MD), a quality functional is evaluated in step O30 for each synthetic dataset SD. The quality functional in this case can be understood as a measure for the match between the statistical characteristics of the original dataset MD and the statistical characteristics of the respective synthetic dataset SD. The greater the match, the better to ability of the respective synthetic dataset SD to be used. In particular the quality functional can be based on a comparison of the statistical distributions between the values for at least one higher-ranking Variable in the synthetic and the original dataset. In other words a statistical distribution of the synthetic values within a synthetic dataset SD is established for this for at least one of the higher-ranking variables in each case and compared with the corresponding statistical distribution of the original values. In accordance with one form of embodiment the quality functional is embodied in such a way that, for each of the higher-ranking variables, a comparative evaluation of the statistical characteristics is undertaken, which are then aggregated. For numerical variables a Kolmogorov-Smirnov test can be applied for example in order to check whether the original and synthetic values of a variable are subject to the same empirical distribution. For categorical variables for example the Chi square test can be used for example. The results for the individual higher-ranking variables can then be aggregated in the quality functional. For example the number of non-refuted null hypotheses can be counted. Since the null hypothesis in mathematical statistics corresponds to the equality of states of affairs, the associated selection value is all the better suited, the larger the result is. As an alternative the significance values (also called p values) can also be added in the quality functional for the matching of the statistical characteristics for each of the higher-ranking variables. Since the significance values represent a measure of evidence for the credibility of the null hypothesis, here too higher values indicate better suited selection parameters.

Then, in step O40, the output values of the quality functional for the individual selection value are compared with one another.

Then, based upon the comparison in O40, in step O50, that selection value with the best output value for the quality functional is established. This selection value is then used as optimized parameter value for the sampling function DSF. The selection value selected thereby is in other words the selection value that optimizes the quality functional, i.e. minimizes or maximizes the quality functional—depending on the definition of the quality functional.

Although the optimization method with the steps O10 to O50 has been described with the aid of only one parameter to be optimized, it is able to be applied in equal measure to sampling functions of DSFs, in which more than one parameter is to be optimized. The above steps are then executed for each combination of possible selection values of the individual parameters to be optimized.

With the previously described method steps synthetic datasets can be created, with which information contained in an original dataset MD is extracted, transported and can be supplied for utilization, without the original dataset MD having to leave the respective local facility A . . . N. In FIG. 7 below schemes are described as to how in this context so-called longitudinal data can be handled. Longitudinal data is in particular time-resolved data and can have variables for in particular time series for individual higher-ranking variables (e.g. blood pressure measurements can be recorded for each patient over time). Such time-resolved data can have a number of individual datasets, which relate to different points in time or were created or recorded at different points in time. Typically an absolute point in time, such as a date for example, is then encoded in the individual data. If this absolute point in time were to be handled in the synthesizing as a usual higher-ranking variable, systematic errors can occur, which can adversely affect the quality of the synthetic data SD. Using the example of a k-nearest neighbors algorithm in this case a great similarity between two original individual datasets can be suggested, only because these have been recorded at a similar absolute point in time. As a result of this original individual datasets can be identified as nearest neighbors, which merely because of the physiologically relevant original values actually would not at all have such a great similarity. The consequence is that the statistical characteristics of the physiologically relevant variables can be reproduced incorrectly during sampling, with correspondingly disadvantageous consequences for the synthetic dataset SD. Moreover the use of absolute points in time can be problematic in some jurisdictions for data protection legislation reasons.

The question now is how to deal with such absolute points in time (or generally with non-physiological variables or variables irrelevant for the utilization). One option lies in taking account of such variables initially for identification and then not taking them into account during sampling of the medical data SD. Precisely in respect of longitudinal data however relevant information can also be lost by this.

A further option is therefore to convert the absolute points in time into relative points in time. In a first step L10 the original individual datasets are initially divided into groups in this case. In this case a group of original individual datasets is defined by them being assigned to the same patient. In other words such a group thus contains one or more original individual datasets that, although they relate to the same patient, were recorded at different absolute points in time however.

In a next step L20 the absolute points in time are then converted in groups into relative time intervals. To this end, for each group, i.e. for each patient, the earliest absolute point in time is determined. This earliest absolute point in time then serves as the reference point, from which the relative time intervals to the other absolute points in time are computed. This can be implemented for example by a subtraction of the earliest absolute point in time from the other absolute points in time of the further original individual datasets of the respective group. The result then gives the relative time interval to the earliest absolute point in time. The original individual dataset or datasets with the earliest absolute points in time are allocated the relative time interval of zero. The time intervals computed in this way can be stored in the associated original individual datasets as further original values. Accordingly a new higher-ranking variable is created in the original dataset MD with the relative time interval.

Building on this, with the steps L30A and L30B, two alternative approaches are provided as to how the relative time intervals are handled during creation of the synthetic dataset SD. In accordance with one form of embodiment a user can select which of the two steps are to be executed. This can take place for example via a user interface in the central unit 100.

In step L30A the relative time interval is simply considered as a further higher-ranking variable, via which there is then regular sampling using the sampling function DSF. Through this conditional probabilities between the relative time interval and the other higher-ranking variables can be preserved. However the synthetic values for the relative time interval are then created based on the complete original dataset MD. Consequently the new relative time intervals are no longer patient-specific or group-specific. For many applications however this is unproblematic, since the loss of information associated therewith is not relevant for many issues arising during utilization.

In order to preserve the aforementioned conditional probabilities, in an alternative approach according to step L30B it is proposed that group-specific or patient-specific sampling be undertaken after the computation of the relative time intervals. During creation of synthetic individual datasets in this case only original individual datasets that belong to the same patient in each case are included. This leads to a synthetic dataset SD with “synthetic patients”, whose associated synthetic individual datasets map the temporal dimension of the original dataset MD. However step L30B demands a sufficient number of individual datasets per group (per patient), since otherwise a group-related sampling of the individual datasets based upon a data pool that is too small occurs, which can lead to statistical artifacts. This is then the case with k-nearest neighbors algorithms for example when the number of the individual datasets lies in the order of magnitude of the number k of nearest neighbors. A further aspect is that a sampling of a number of original individual datasets or a group that is too small can lead to synthetic datasets SD that are too “similar” to the original data MD. Accordingly there is the danger of a reconstruction of personal data. In an optional substep L31B there is therefore provision for there to be a check on the original dataset MD as to whether this is suitable for patient-related sampling. If not, this can be notified accordingly to a user in the central unit 100.

In conclusion it is pointed out that the methods described in detail here and the apparatuses presented merely involve example embodiments, which can be modified by the person skilled in the art in a wide diversity of ways, without departing from the field of the invention. Furthermore the use of the indefinite article “a” or “an” does not exclude the features concerned also being able to be present multiple times. Likewise the terms “unit”, “facility” and “element” do not exclude the components concerned being able to consist of a number of components working together, which if necessary can also be physically distributed.

The following points are likewise part of the disclosure:
1. A computer-implemented method for creating a synthetic dataset (SD) based on a medical dataset (MD), wherein the method has the following steps:

provision (S10) of a medical dataset (MD), which has a number of original individual datasets (RDS1 . . . RDS6), which are assigned to real existing patients and have original values for one or more higher-ranking variables (x, y);

creation (S20) of a synthetic dataset (SD) based on the medical dataset (MD), wherein the synthetic dataset (SD) has a number of synthetic individual datasets (SDS), which have synthetic values for at least some of the higher-ranking variables (x, y) of the medical dataset (MD), but cannot be related back to a real existing patient;

wherein, in the step of creation (S30), the synthetic dataset (SD) is created by applying a sampling function (DSF) to the medical dataset (MD), which sampling function (DSF) is embodied to create the synthetic dataset (SD) by sampling the entire medical dataset (MD) while replacing all original values.
2. The method according to 1, in which the sampling function (DSF) has a trained function.
3. The method according to one of the preceding points, in which the sampling function (DSF) has a k-nearest neighbors algorithm.
4. The method according to one of the preceding points, in which, in the step of creation (S20), the synthetic values of each synthetic individual dataset (SDS) are computed in each case based on original values of a number of original individual datasets (RDS1 . . . RDS6).
5. The method according to one of the preceding points, in which:

in the medical dataset (MD) a number of data classes are defined and each original individual dataset (RDS1 . . . RDS6) is assigned to a data class; and

in the step of creation (S20) the sampling function (DSF) is applied to each of the data classes separately, so that for each data class synthetic datasets (SDS) are created based on only the original individual datasets (RDS1 . . . RDS6) assigned to the data class.
6. The method according to 5, further with the step:

definition (D12) of the number of data classes in the medical dataset (MD).
7. The method according to 5 or 6, in which a first of the data classes has a first number of original individual datasets (RDS1 . . . RDS6) and a second of the data classes has a second number of original individual datasets (RDS1 . . . RDS6), wherein the first number is smaller than the second number.
8. The method according to one of the preceding points, in which the number of the synthetic individual datasets (SDS) in the synthetic dataset (SD) is greater than the number of the original individual datasets in the medical dataset (MD).
9. The method according to one of the preceding points, further with the step:

computation (O30) of a quality functional, which quality functional is a measure for the match between the statistical characteristics of the synthetic dataset (SD) and the statistical characteristics of the original dataset (MD).
10. The method according to 9, in which, for the

at least one parameter (k), the sampling function (DSF) is optimized (O10-O40) by optimizing the quality functional for the medical dataset (MD).
11. The method according to 10, in which the optimization comprises:

definition (O10) of a number of selection values for the parameter (k);

creation (O20) of a synthetic dataset (SD) in each case for each of the number of selection values, wherein the respective selection value is used as a value for the parameter (k) of the sampling function (DSF) to be optimized;

computation (O30) of the quality functional for each created synthetic dataset (SD),

comparison (O40) of the computed quality functionals;

selection (O50) of an optimal selection value for the parameter (k) to be optimized, based on the comparison.
12. The method according to one of the preceding points, further with the step:

selection (D14) of variables to be sampled from the higher-ranking variables (x, y), wherein

in the step of creation (S20), the sampling function (DSF) is only applied to the original values of the medical dataset (MD) that belong to the selected variables to be sampled, so that the synthetic dataset (SD) merely has synthetic values for the variables to be sampled.
13. The method according to one of the preceding points, in which:

one of the higher-ranking variables (x, y) refers to an absolute point in time at which the original values of an original individual dataset were recorded;

further with the step of converting the absolute points in time into relative time intervals, wherein
the relative time intervals are each defined within groups of the original individual datasets (RDS1 . . . RDS6), which groups are defined by the assignment of the original individual datasets (RDS1 . . . RDS6) to the same patient, and
the earliest absolute point in time within a group is used as a reference time for computing the relative time intervals.
14. The method according to one of the preceding points, in which

in the step of creation, for the creation of a synthetic individual dataset (SDS) only those original individual datasets (RDS1 . . . RDS6) that belong to the same patient are sampled in each case.
15. A computer-implemented method for utilization of a medical dataset (MD), wherein the medical dataset (MD) is stored locally within a first facility (A) and has a number of original individual datasets (RDS1 . . . RDS6), which are assigned to real existing patients and have original values for one or more higher-ranking variables (x, y);

creation (S20) of a synthetic dataset (SD) based on the medical dataset (MD), wherein the synthetic dataset (SD) has a number of synthetic individual datasets (SDS), which have synthetic values for the same higher-ranking variables (x, y) as the medical dataset (MD), but cannot be related back to an original existing patient, wherein the step of creation (S20) is undertaken locally within the first facility (A) by application of sampling function (DSF) to the medical data (MD);

transfer (S30) of the synthetic dataset (SD) from the first facility (A) to a central unit (100) outside the first facility (A);

utilization of (S40) the synthetic dataset (SD) within the central unit (100).
16. The method according to 15, wherein the step of utilization (S40) comprises:

training of a trainable classifier to predict a clinical outcome based on the synthetic dataset (SD), and/or

validation of a trainable classifier to predict a clinical outcome based on the synthetic dataset (SD), and/or

a statistical evaluation of the synthetic dataset (SD), and/or

archiving of the synthetic dataset (SD) in the central unit (100).
17. The method according to 15 or 16, further with the step:

provision (S21) of a sampling function (DSF) in the first facility (A), which sampling function (DSF) is embodied for creating the synthetic dataset (SD).
18. The method according to one of points 15 to 17,

in which the step of creation features a method of claims 1 to 14.
19. A system (1) for utilization of a medical dataset (MD), wherein

the medical dataset (MD) is stored locally in a first facility (A) and has a number of original individual datasets (RDS1 . . . RDS6), which are assigned to real existing patients and have original values for one or more higher-ranking variables (x, y);

the system has a computing unit (110) outside the first facility (A) and an interface for communication between the computing unit (110) and the first facility (A);

the computing unit (110) is embodied:
to induce a local creation (S20) of a synthetic dataset (SD) in the first facility (A) via the interface, which synthetic dataset (SD) has a number of synthetic individual datasets (SDS), which have synthetic values for the same higher-ranking variables (x, y) as the medical dataset (MD), but cannot be related back to a real existing patient;
to receive the synthetic dataset (MD) from the first facility (A) via the interface; and
to utilize the synthetic dataset (MD) outside the first facility (A).

Of course, the embodiments of the method according to the invention and the imaging apparatus according to the invention described here should be understood as being example. Therefore, individual embodiments may be expanded by features of other embodiments. In particular, the sequence of the method steps of the method according to the invention should be understood as being example. The individual steps can also be performed in a different order or overlap partially or completely in terms of time.

The patent claims of the application are formulation proposals without prejudice for obtaining more extensive patent protection. The applicant reserves the right to claim even further combinations of features previously disclosed only in the description and/or drawings.

References back that are used in dependent claims indicate the further embodiment of the subject matter of the main claim by way of the features of the respective dependent claim; they should not be understood as dispensing with obtaining independent protection of the subject matter for the combinations of features in the referred-back dependent claims. Furthermore, with regard to interpreting the claims, where a feature is concretized in more specific detail in a subordinate claim, it should be assumed that such a restriction is not present in the respective preceding claims.

Since the subject matter of the dependent claims in relation to the prior art on the priority date may form separate and independent inventions, the applicant reserves the right to make them the subject matter of independent claims or divisional declarations. They may furthermore also contain independent inventions which have a configuration that is independent of the subject matters of the preceding dependent claims.

None of the elements recited in the claims are intended to be a means-plus-function element within the meaning of 35 U.S.C. §112(f) unless an element is expressly recited using the phrase “means for” or, in the case of a method claim, using the phrases “operation for” or “step for.”

Example embodiments being thus described, it will be obvious that the same may be varied in many ways. Such variations are not to be regarded as a departure from the spirit and scope of the present invention, and all such modifications as would be obvious to one skilled in the art are intended to be included within the scope of the following claims.

What is claimed is:

1. A computer-implemented method for a medical dataset, comprising:
storing the medical dataset within a first facility, the medical dataset including a number of original individual datasets assigned to real existing patients and including original values for one or more higher-ranking variables;
creating a synthetic dataset based on the medical dataset, each synthetic individual dataset of the number of synthetic individual datasets including synthetic values for same higher-ranking variables as the one or more higher-ranking variables medical dataset, not relating back to a real existing patient, wherein the creating is undertaken within the first facility by application of a sampling function to the medical data; and
transferring the synthetic dataset from the first facility to a central unit outside the first facility, the synthetic dataset being utilizable within the central unit.

2. The computer-implemented method of claim 1, wherein the sampling function is embodied to create the synthetic dataset by sampling the entire medical dataset while replacing all the original values.

3. The computer-implemented method of claim 1, wherein the sampling function includes a trained function.

4. The computer-implemented method of claim 1, wherein the sampling function includes a k-nearest neighbors algorithm.

5. The computer-implemented method of claim 1, wherein:
a number of data classes are defined in the medical dataset and wherein each respective original individual dataset, of the number of original individual datasets assigned to real existing patients, is assigned to a respective data class of the number of data classes; and
wherein in the creating, the sampling function is applied to each of the number of data classes separately, so that each respective data class synthetic datasets are created during the creating, based on only a respective original individual dataset assigned to the respective data class.

6. The computer-implemented method of claim 1, wherein the synthetic dataset is utilizable within the central unit for at least one of:
training of a trainable classifier to predict a clinical outcome based on the synthetic dataset;
validation of a trainable classifier to predict a clinical outcome based on the synthetic dataset;
a statistical evaluation of the synthetic dataset; and
archiving of the synthetic dataset in the central unit.

7. The computer-implemented method of claim 1, further comprising:
provisioning of a sampling function in the first facility, the sampling function being embodied for creating the synthetic dataset.

8. The computer-implemented method of claim 1, wherein the number of the synthetic individual datasets in the synthetic dataset is greater than the number of the original individual datasets in the medical dataset.

9. The computer-implemented method of claim 1, further comprising:
computing a quality functional, the quality functional being a measure for the match between the statistical characteristics of the synthetic dataset and the statistical characteristics of the original dataset.

10. The computer-implemented method of claim 9, wherein at least one parameter of the sampling function is optimized by optimizing the quality functional for the medical dataset.

11. The computer-implemented method of claim 10, wherein the optimizing comprises:
defining a number of selection values for the parameter;
creating a respective synthetic dataset for each respective selection value of the number of selection values, wherein the respective selection value is used as the value for the parameter of sampling function to be optimized;
computing the quality functional for each respective synthetic dataset created;
comparing the computed quality functionals; and
selecting an optimal selection value for the parameter to be optimized based on the comparing.

12. The computer-implemented method of claim 1, further comprising:
selecting variables to be sampled from the higher-ranking variables, wherein
in the creating, the sampling function is only applied to the original values of the medical dataset belonging to the variables to be sampled, so that the synthetic dataset includes synthetic values for the variables to be sampled.

13. The computer-implemented method of claim 1, wherein
one of the higher-ranking variables refers to an absolute point in time, in which the original values of an original individual dataset were recorded; and the computer-implemented method further comprising:
converting the absolute points in time into relative time intervals, wherein
the relative time intervals are each defined within groups of the original individual datasets defined by assignment of the original individual datasets to the same patient, and
a relatively earliest absolute point in time within a group is used as a reference time for computing the relative time intervals.

14. The computer-implemented method of claim 1, wherein in the creating, for creation of a respective synthetic individual dataset, only original individual datasets belonging to the same patient are sampled.

15. A system for a medical dataset, a first facility storing the medical dataset, the medical dataset including a number of original individual datasets assigned to real existing patients and including original values for one or more higher-ranking variables, the system comprising:
a computing unit, located outside the first facility; and
an interface for communication between the computing unit and the first facility, wherein
the computing unit is embodied:
to induce a local creation of a synthetic dataset in the first facility via the interface, the synthetic dataset including a number of synthetic individual datasets, each synthetic individual dataset of the number of synthetic individual datasets including synthetic values for same higher-ranking variables as the one or more higher-ranking variables medical dataset, not relating back to a real existing patient;
to receive the synthetic dataset from the first facility via the interface; and
to utilize the synthetic dataset outside the first facility.

16. A non-transitory computer program product, including a program, directly loadable into a memory of a programmable computing unit of a processing unit, the program including program segments for carrying out the method of claim 1 when the program is executed in the computing unit of the processing unit.

17. A non-transitory computer-readable memory medium, storing readable and executable program sections for carrying out the method of claim 1 when the program sections are executed by at least one of a determination system and training system.

18. The computer-implemented method of claim 2, wherein the sampling function includes a trained function.

19. The computer-implemented method of claim 2, wherein the sampling function includes a k-nearest neighbors algorithm.

20. The computer-implemented method of claim 2, wherein:
a number of data classes are defined in the medical dataset and wherein each respective original individual dataset, of the number of original individual datasets assigned to real existing patients, is assigned to a respective data class of the number of data classes; and
wherein in the creating, the sampling function is applied to each of the number of data classes separately, so that each respective data class synthetic datasets are created during the creating, based on only a respective original individual dataset assigned to the respective data class.

21. The computer-implemented method of claim 2, wherein the synthetic dataset is utilizable within the central unit for at least one of:
training of a trainable classifier to predict a clinical outcome based on the synthetic dataset;
validation of a trainable classifier to predict a clinical outcome based on the synthetic dataset;
a statistical evaluation of the synthetic dataset; and
archiving of the synthetic dataset in the central unit.

22. The computer-implemented method of claim 2, further comprising:
provisioning of a sampling function in the first facility, the sampling function being embodied for creating the synthetic dataset.

23. The computer-implemented method of claim 2, wherein the number of the synthetic individual datasets in the synthetic dataset is greater than the number of the original individual datasets in the medical dataset.

24. The computer-implemented method of claim 2, further comprising:
computing a quality functional, the quality functional being a measure for the match between the statistical characteristics of the synthetic dataset and the statistical characteristics of the original dataset.

25. The computer-implemented method of claim 24, wherein at least one parameter of the sampling function is optimized by optimizing the quality functional for the medical dataset.

26. The computer-implemented method of claim 25, wherein the optimizing comprises:
defining a number of selection values for the parameter;
creating a respective synthetic dataset for each respective selection value of the number of selection values, wherein the respective selection value is used as the value for the parameter of sampling function to be optimized;
computing the quality functional for each respective synthetic dataset created;
comparing the computed quality functionals; and
selecting an optimal selection value for the parameter to be optimized based on the comparing.

27. The computer-implemented method of claim 2, further comprising:
selecting variables to be sampled from the higher-ranking variables, wherein
in the creating, the sampling function is only applied to the original values of the medical dataset belonging to the variables to be sampled, so that the synthetic dataset includes synthetic values for the variables to be sampled.

28. The computer-implemented method of claim 2, wherein
one of the higher-ranking variables refers to an absolute point in time, in which the original values of an original individual dataset were recorded; and the computer-implemented method further comprising:
converting the absolute points in time into relative time intervals, wherein
the relative time intervals are each defined within groups of the original individual datasets defined by assignment of the original individual datasets to the same patient, and
a relatively earliest absolute point in time within a group is used as a reference time for computing the relative time intervals.

29. The computer-implemented method of claim 2, wherein in the creating, for creation of a respective synthetic individual dataset, only original individual datasets belonging to the same patient are sampled.
